(19) United States
(12) Patent Application Publication
(10) Pub. No.: US 2025/0378966 A1
(43) Pub. Date: Dec. 11, 2025

(54) System, Method, and Device for Determining Hyperactivity Based on Sensor Data

(71) Applicants: Sam Shaaban, Pittsburgh, PA (US); Carnegie Mellon University, Pittsburgh, PA (US); University of Pittsburgh - Of the Commonwealth System of Higher Education, Pittsburgh, PA (US)

(72) Inventors: Mayank Goel, Pittsburgh, PA (US); Oliver Lindhiem, Pittsburgh, PA (US); Sam Shaaban, Pittsburgh, PA (US)

(21) Appl. No.: 19/234,794

(22) Filed: Jun. 11, 2025

Related U.S. Application Data
(60) Provisional application No. 63/658,488, filed on Jun. 11, 2024.

(51) Int. Cl. G16H 50/30; A61B 5/00; A61B 5/024; A61B 5/11

(52) U.S. Cl. CPC G16H 50/30; A61B 5/02438; A61B 5/11; A61B 5/6801

(57) ABSTRACT

Provided is a system, method, and device for determining hyperactivity based on sensor data. The system includes at least one processor configured to collect sensor data from a wearable device worn by a subject user over a time period, the sensor data comprising at least motion data for the subject user, extract features from the sensor data, automatically assign at least one activity label of a plurality of activity labels to each feature based on at least one classification model, apply context filtering to the features based on the plurality of activity labels resulting in filtered feature data, and generate a hyperactivity risk score for the subject user based on at least one machine-learning model and the filtered feature data.

### CROSS REFERENCE TO RELATED APPLICATION

This application claims the benefit of U.S. Provisional Patent Application No. 63/658,488 filed on Jun. 11, 2024, the disclosure of which is hereby incorporated by reference in its entirety.

### GOVERNMENT SUPPORT

This invention was made with United States government support under MH119644 awarded by the National Institutes of Health. The U.S. government has certain rights in the invention.

### BACKGROUND OF THE INVENTION

### Field

This disclosure relates generally to sensors and, in non-limiting embodiments, to systems, methods, and devices for determining hyperactivity in a subject based on sensor data.

### Technical Considerations

The current methods for measuring hyperactivity in children rely on parents' or teachers' reports, which may be vulnerable to subjectivity.

### SUMMARY

According to non-limiting embodiments or aspects, provided is a system comprising: at least one processor configured to: collect sensor data from a wearable device worn by a subject user over a time period, the sensor data comprising at least motion data for the subject user; extract features from the sensor data; automatically assign at least one activity label of a plurality of activity labels to each feature based on at least one classification model; apply context filtering to the features based on the plurality of activity labels resulting in filtered feature data; and generate a hyperactivity risk score for the subject user based on at least one machine-learning model and the filtered feature data. In non-limiting embodiments or aspects, the at least one processor is further configured to train the machine-learning model based on the filtered feature data.

In non-limiting embodiments or aspects, the at least one processor is further configured to: determine the hyperactivity risk score for each time segment of a plurality of time segments of the time period, resulting in a plurality of risk scores; and combine the plurality of risk scores to generate a daily hyperactivity risk score for the subject user. In non-limiting embodiments or aspects, the at least one processor is further configured to display at least one graphical user interface configured to visualize the hyperactivity risk score across different contexts and time segments based on user interaction. In non-limiting embodiments or aspects, the sensor data comprises the motion data and at least one of location data and heart rate data. In non-limiting embodiments or aspects, the at least one processor comprises a processor of the wearable device. In non-limiting embodiments or aspects, the at least one processor comprises a processor of a separate computing device.

According to non-limiting embodiments or aspects, provided is a method for detecting hyperactivity in a subject user, comprising: collecting sensor data from a wearable device worn by the subject user over a time period, the sensor data comprising at least motion data for the subject user; extracting features from the sensor data; automatically assigning at least one activity label of a plurality of activity labels to each feature based on at least one classification model; applying context filtering to the features based on the plurality of activity labels resulting in filtered feature data; and generating a hyperactivity risk score for the subject user based on at least one machine-learning model and the filtered feature data. In non-limiting embodiments or aspects, the method further includes: training the machine-learning model based on the filtered feature data. In non-limiting embodiments or aspects, the method further includes: determining the hyperactivity risk score for each time segment of a plurality of time segments of the time period, resulting in a plurality of risk scores; and combining the plurality of risk scores to generate a daily hyperactivity risk score for the subject user. In non-limiting embodiments or aspects, the method further includes: displaying at least one graphical user interface configured to visualize the hyperactivity risk score across different contexts and temporal segments based on interaction with a user. In non-limiting embodiments or aspects, the sensor data comprises the motion data and at least one of location data and heart rate data. In non-limiting embodiments or aspects, collecting the sensor data comprises collecting the sensor data using a processor of the wearable device. In non-limiting embodiments or aspects, collecting the sensor data comprises collecting the sensor data using a processor of a separate computing device.

According to non-limiting embodiments or aspects, provided is a computer program product comprising at least one non-transitory computer-readable medium including program instructions that, when executed by at least one processor, cause the at least one processor to: collect sensor data from a wearable device worn by a subject user, the sensor data comprising at least motion data for the subject user over a period of time; extract features from the sensor data; automatically assign at least one activity label of a plurality of activity labels to each feature based on at least one classification model; apply context filtering to the features based on the plurality of activity labels resulting in filtered feature data; and generate a hyperactivity risk score for the subject user based on at least one machine-learning model and the filtered feature data.

Other preferred and non-limiting embodiments or aspects of the present invention will be set forth in the following numbered clauses:

Clause 1: A system comprising: at least one processor configured to: collect sensor data from a wearable device worn by a subject user over a time period, the sensor data comprising at least motion data for the subject user; extract features from the sensor data; automatically assign at least one activity label of a plurality of activity labels to each feature based on at least one classification model; apply context filtering to the features based on the plurality of activity labels resulting in filtered feature data; and generate a hyperactivity risk score for the subject user based on at least one machine-learning model and the filtered feature data.

Clause 2: The system of clause 1, wherein the at least one processor is further configured to train the machine-learning model based on the filtered feature data.

Clause 3: The system of any of clauses 1-2, wherein the at least one processor is further configured to: determine the hyperactivity risk score for each time segment of a plurality of time segments of the time period, resulting in a plurality of risk scores; and combine the plurality of risk scores to generate a daily hyperactivity risk score for the subject user.

Clause 4: The system of any of clauses 1-3, wherein the at least one processor is further configured to display at least one graphical user interface configured to visualize the hyperactivity risk score across different contexts and time segments based on user interaction.

Clause 5: The system of any of clauses 1-4, wherein the sensor data comprises the motion data and at least one of location data and heart rate data.

Clause 6: The system of any of clauses 1-5, wherein the at least one processor comprises a processor of the wearable device.

Clause 7: The system of any of clauses 1-6, wherein the at least one processor comprises a processor of a separate computing device.

Clause 8: A method for detecting hyperactivity in a subject user, comprising: collecting sensor data from a wearable device worn by the subject user over a time period, the sensor data comprising at least motion data for the subject user; extracting features from the sensor data; automatically assigning at least one activity label of a plurality of activity labels to each feature based on at least one classification model; applying context filtering to the features based on the plurality of activity labels resulting in filtered feature data; and generating a hyperactivity risk score for the subject user based on at least one machine-learning model and the filtered feature data.

Clause 9: The method of clause 8, further comprising: training the machine-learning model based on the filtered feature data.

Clause 10: The method of any of clauses 8-9, further comprising: determining the hyperactivity risk score for each time segment of a plurality of time segments of the time period, resulting in a plurality of risk scores; and combining the plurality of risk scores to generate a daily hyperactivity risk score for the subject user.

Clause 11: The method of any of clauses 8-10, further comprising: displaying at least one graphical user interface configured to visualize the hyperactivity risk score across different contexts and temporal segments based on interaction with a user.

Clause 12: The method of any of clauses 8-11, wherein the sensor data comprises the motion data and at least one of location data and heart rate data.

Clause 13: The method of any of clauses 8-12, wherein collecting the sensor data comprises collecting the sensor data using a processor of the wearable device.

Clause 14: The method of any of clauses 8-13, wherein collecting the sensor data comprises collecting the sensor data using a processor of a separate computing device.

Clause 15: A computer program product comprising at least one non-transitory computer-readable medium including program instructions that, when executed by at least one processor, cause the at least one processor to: collect sensor data from a wearable device worn by a subject user, the sensor data comprising at least motion data for the subject user over a period of time; extract features from the sensor data; automatically assign at least one activity label of a plurality of activity labels to each feature based on at least one classification model; apply context filtering to the features based on the plurality of activity labels resulting in filtered feature data; and generate a hyperactivity risk score for the subject user based on at least one machine-learning model and the filtered feature data.

Clause 16: The computer program product of clause 15, wherein the at least one processor is further caused to train the machine-learning model based on the filtered feature data.

Clause 17: The computer program product of any of clauses 15-16, wherein the at least one processor is further caused to: determine the hyperactivity risk score for each time segment of a plurality of time segments of the time period, resulting in a plurality of risk scores; and combine the plurality of risk scores to generate a daily hyperactivity risk score for the subject user.

Clause 18: The computer program product of any of clauses 15-17, wherein the at least one processor is further caused to display at least one graphical user interface configured to visualize the hyperactivity risk score across different contexts and time segments based on user interaction.

Clause 19: The computer program product of any of clauses 15-18, wherein the sensor data comprises the motion data and at least one of location data and heart rate data.

Clause 20: The computer program product of any of clauses 15-19, wherein the at least one processor comprises a processor of the wearable device.

These and other features and characteristics of the present disclosure, as well as the methods of operation and functions of the related elements of structures and the combination of parts and economics of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

Additional advantages and details are explained in greater detail below with reference to the exemplary embodiments that are illustrated in the accompanying schematic figures in which:

FIG. 1 is a schematic diagram of a system for determining hyperactivity in a subject based on sensor data according to non-limiting embodiments or aspects;

FIG. 2 is another schematic diagram of a system for determining hyperactivity in a subject based on sensor data according to non-limiting embodiments or aspects;

FIG. 3 is a flow diagram of a method for determining hyperactivity in a subject based on sensor data according to non-limiting embodiments or aspects;

FIG. 4A-4C illustrate graphical user interfaces used with a system for determining hyperactivity in a subject based on sensor data according to non-limiting embodiments or aspects;

FIG. 5 illustrates another graphical user interface used with a system for determining hyperactivity in a subject based on sensor data according to non-limiting embodiments or aspects; and

FIG. 6 illustrates example components of a computing device used in connection with non-limiting embodiments.

### DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the invention as it is oriented in the drawing figure. However, it is to be understood that the invention may assume various alternative variations and step sequences, except where expressly specified to the contrary. It is also to be understood that the specific devices and processes illustrated in the attached drawing, and described in the following specification, are simply exemplary embodiments or aspects of the invention. Hence, specific dimensions and other physical characteristics related to the embodiments or aspects disclosed herein are not to be considered as limiting.

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the terms “communication” and “communicate” refer to the receipt or transfer of one or more signals, messages, commands, or other type of data. For one unit (e.g., any device, system, or component thereof) to be in communication with another unit means that the one unit is able to directly or indirectly receive data from and/or transmit data to the other unit. This may refer to a direct or indirect connection that is wired and/or wireless in nature. Additionally, two units may be in communication with each other even though the data transmitted may be modified, processed, relayed, and/or routed between the first and second unit. For example, a first unit may be in communication with a second unit even though the first unit passively receives data and does not actively transmit data to the second unit. As another example, a first unit may be in communication with a second unit if an intermediary unit processes data from one unit and transmits processed data to the second unit. It will be appreciated that numerous other arrangements are possible.

As used herein, the term “computing device” may refer to one or more devices configured to process data. A computing device may, in some examples, include the necessary components to receive, process, and output data, such as a processor, a display, a memory, an input device, a network interface, and/or the like. A computing device may be a processor, such as a CPU or GPU, a mobile device, and/or other like devices. A computing device may also be a desktop computer, a server computer or other form of non-mobile computer. Reference to “a processor,” as used herein, may refer to a previously-recited processor that is recited as performing a previous step or function, a different processor, and/or a combination of processors. For example, as used in the specification and the claims, a first processor that is recited as performing a first step or function may refer to the same or different processor recited as performing a second step or function.

Non-limiting embodiments described herein provide a method, system, and device by which the hyperactivity in an individual may be measured by collecting sensor data from a wearable device worn by a user over a period of time (e.g., hours, a day, a week, a month, and/or the like) and extracting features from the sensor data including, but not limited to, sleep, body motion data, location data, and heart rate. The wearable device may be, for example, a smartwatch that passively collects health data about the user in their daily life. It will be appreciated that other wearable devices and/or sensor arrangements may be used. A wearable device may include sensors such as one or more accelerometers, gyroscopes, heartrate sensors, location sensors (e.g., GPS or the like), and/or other like sensors.

In non-limiting embodiments, a Fast Fourier Transform (FFT) may be applied to each feature. It will be appreciated that other transformations may be used in non-limiting embodiments. Activity labels may be automatically assigned, without human input, to each of the features acquired from the sensor based on one or more pre-trained classification machine-learning models (e.g., for example trained on human-created labels). The pre-trained classification machine-learning model may be configured to apply a filter that determines circumstances where the difference between users with and without hyperactivity would be more significant based on sensor data, such as but not limited to location data, heart rate sensor data, and motion data (e.g., processed according to motion-based human activity recognition models). In this manner, activity labels may be predicted and/or estimated based on multiple types of input data, including sensor data. The feature set is input into the filter and the resulting filtered feature data is input into a machine-learning model for training, which subsequently produces activity labels (e.g., classifications) and generates an objective plurality of risk scores that is combined to generate a daily hyperactivity risk for the user. It will be appreciated that the risk score may be calculated for any time period, such as a part of a day, a day, multiple days, a week, a month, a year, and/or the like.

In non-limiting embodiments, the resulting plurality of hyperactivity risk scores is combined to generate a cumulative hyperactivity risk score for the time period for the target user. The risk score may have a range score (e.g., 0-1, 1-10, 1-100, and/or the like), rather than binary (e.g., hyperactive risk or not). This allows for a granularity that is not available with binary classification methods. In non-limiting embodiments, the cumulative hyperactivity risk score may be used to determine medication dosages, changes in medication, and/or the like.

Non-limiting embodiments described herein provide a graphical user interface (GUI) that is configured to visualize the target user's plurality of risk scores across different contexts in addition to the user's daily hyperactivity risk. The GUI may include one or more tools and/or selectable options to be interacted with by a clinician, healthcare professional, guardian, and/or other like user. The GUI may allow for data to be viewed over different time periods and for comparisons. In non-limiting embodiments, the GUI may be used to generate a risk score for a selected time period, such as a per day risk score. In non-limiting embodiments, the GUI may allow a user to selectively filter the risk scores and/or sensor data by location and/or activity, and to interact with the risk scores and/or sensor data to alter the variables and/or time ranges used to generate the score(s).

Non-limiting embodiments described herein may be implemented on a wearable device and/or on a remote server computer. For example, in non-limiting embodiments a smartwatch or other wearable device may execute one or more machine-learning models to label and/or classify activities and/or to generate one or more risk scores. This allows for real-time (e.g., nearly immediate) feedback with scores and/or context information.

In non-limiting embodiments, the smartwatch and/or another computing device may automatically implement interventions (e.g., “Just In Time” interventions). For example, an algorithm may compare real-time risk scores (e.g., risk-scores representing the subject within seconds or minutes) with one or more thresholds to determine a high-risk situation, in response to which the subject user and/or a guardian or caregiver may be alerted. For example, a notification may be communicated to a guardian's device and/or an application on the wearable device worn by the subject user, and the notification may prompt the wearer to perform an activity or engage with device in some manner.

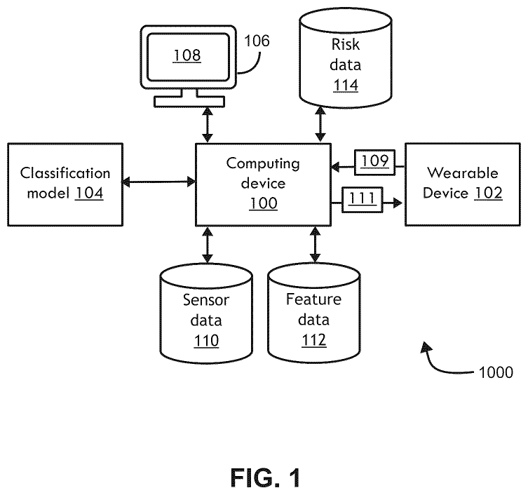

Referring to FIG. 1, shown is a schematic diagram showing a system 1000 for determining hyperactivity based on sensor data according to non-limiting embodiments or aspects. For example, the system 1000 may measure hyperactivity in an individual by collecting sensor data 109 from a wearable device 102, such as but not limited to a watch with sensors, worn by the user over a period of time (e.g., hours, a day, a week, a month, and/or the like) and extracting features from the sensor data including, but not limited to, sleep, body motion data, location data, and heart rate. In some examples a mobile phone carried by the user may be used as the wearable device. A computing device 100 may include at least one processor configured to send and receive information to and from the wearable device 102. The computing device 100 may be local or remote from the wearable device 102. The computing device 100 is in communication with a wearable device 102. The communication between the computing device may be a Bluetooth communication via an application installed on the wearable device 102. In some examples, the wearable device 102 may be in communication with a local computing device that is in communication with a remote server computer (e.g., such as computing device 100) via a network. In some examples, the wearable device may be in communication with a remote server computer (e.g., such as computing device 100) via a network, such as wireless Internet, one or more cellular or mobile data networks, and/or the like. It will be appreciated that various configurations are possible.

With continued reference to FIG. 1, the sensor data 109 received may include raw sensor data, such as accelerometer, heart rate, geolocation, and/or gyroscope measurements associated with time, and/or may include processed sensor data that may convert raw measurements with thresholds and/or algorithms. Non-limiting embodiments combine motion data (such as accelerometer data) in combination with multiple other forms of data (e.g., geolocation, heart rate, etc.) to provide a robust data set. The sensor data 109 may be stored in one or more data storage devices as sensor data 110 in communication with the computing device. The computing device 100 or another computing device may extract features from the sensor data 110, which may include mathematical representations (e.g., such as vectors) of sensor data parameters such as movement, heartrate, trends, and/or the like. The features may be stored as feature data 112 in one or more data storage devices.

With continued reference to FIG. 1, the computing device 100 may also include and/or be in communication with a classification model 104, which may include one or more machine-learning models. In non-limiting embodiments, the computing device 100 or another computing device trains the classification model 104 based on feature data 112 extracted from the sensor data 110. In-non-limiting embodiments, the computing device 100 is configured to automatically assign at least one activity label (e.g., classification) from a plurality of activity labels to each feature in the feature data 112 based on the classification model 104. In non-limiting embodiments, the features may be extracted for each time period of a plurality of time periods (e.g., day, afternoon, evening). In non-limiting embodiments, the computing device 100 is configured to apply context filtering to the feature data 112 based on a plurality of activity labels, resulting in a filtered version of feature data 112. The activity labels may be provided through supervision and/or predicted based on the sensor data and a machine-learning model (e.g., model 104 or another model). The activity labels may correspond to different types of activity, such as but not limited to exercise, sitting/quiet, household/daily activities, school, and/or the like. Non-limiting embodiments employ a multi-level process by adding context information after the features are extracted from the sensor data to provide relevant information to the machine learning model generating the risk score.

With continued reference to FIG. 1, the computing device 100 may be configured to generate a daily hyperactivity risk score for the subject user wearing the wearable device 102 based on the classification model 104 and the filtered version of feature data 112. This daily hyperactivity risk score may be determined based on the hyperactivity risk score for each time segment of a plurality of time segments of the time period over which the subject user of the wearable device 102 is being monitored. The resulting plurality of hyperactivity risk scores may then be combined to generate a daily hyperactivity risk score for the subject user. It will be appreciated that, additionally or alternatively to a daily hyperactivity score, different time intervals may be assigned hyperactivity scores, such as morning, afternoon, evening, weekly,

With continued reference to FIG. 1, the computing device 100 may be in communication with another computing device 106, which may include a clinician computer (e.g., a doctor, nurse, or other practitioner), a parent/guardian computer (e.g., a personal computer or mobile device of a parent guardian), or the like. The computing device 106 displays a GUI 108 visualizing the hyperactivity risk scores across different contexts and time based on user interaction by a practitioner or other individual (e.g., parent, guardian, teacher, counselor, and/or the like). In non-limiting embodiments, an alert and/or intervention request may be automatically generated by computing device 100 and communicated to computing device 106 in response to a score satisfying a threshold. For example, an intervention request may be sent to a parent/guardian if a real-time risk score satisfies a high-risk threshold.

Still referring to FIG. 1, the computing device 100 may generate and output feedback 111 that is received by the wearable device 102. The feedback 111 may include scores, prompts for information, prompts for actions or intervention (e.g., rest, change activity, contact a parent/guardian), and/or other data that may be visually and/or audibly presented to the user of the wearable device 102.

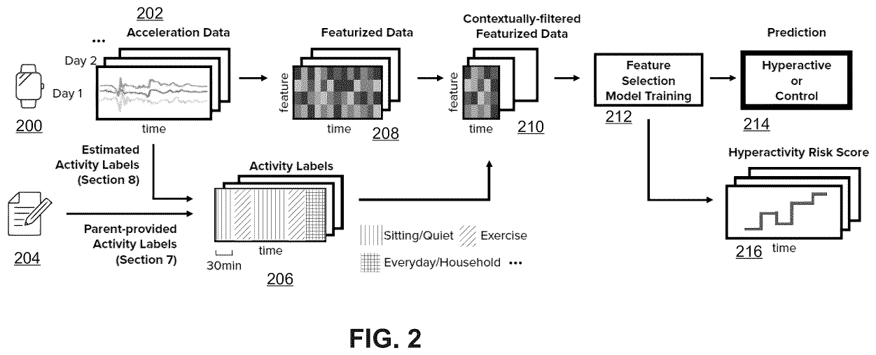

Referring now to FIG. 2, shown is another schematic diagram of a system for determining hyperactivity in a subject based on sensor data according to non-limiting embodiments or aspects. FIG. 2 shows an overview of the machine learning pipeline for estimating the hyperactivity risk scores for uses. In non-limiting embodiments, the machine learning pipeline includes featurization, context filtering, feature selection, and predicting daily risk.

With continued reference to FIG. 2, the inputs to the machine learning pipeline may include acceleration data and activity labels, wherein the acceleration data is measured using the wearable device 200 worn by the user subject (e.g., such as one or more accelerometers thereof). The activity labels 206 may be based on labels 204 originally provided by an entity (e.g., parents or guardians, physicians, and/or the like) of the user/subject to contextualize motion data, and the provided labels 204 may be used to first test the effectiveness of using contextual data. In some non-limiting embodiments, estimated activity labels may be made without relying on provided data to case this burden and may be a fully automated pipeline. In some examples, and as shown in FIG. 2, estimated activity labels may be determined based on the acceleration data 202 and combined with the provided activity labels 204 to form activity data 206 showing labels according to time. In non-limiting embodiments, the contextual data may be used for each half hour time window (slot), although various sized time windows may be used. In FIG. 2, the activity labels include sitting/quiet, exercise, and everyday/household, referring to three categories of movement. It will be appreciated that additional labels may be used to further differentiate between different levels of activity.

With continued reference to FIG. 2, the approach to using provided activity labels 204 requires the acceleration data, which includes movement in three axes (x,y,z) sampled at a rate of, for example, 50 Hz. In non-limiting embodiments, several features may be extracted to form featurized data 208 including different time window sizes to capture various signal characteristics, wherein the used window sizes are 5 s, 60 s, and 600 s. In non-limiting embodiments, for each used window size, the maximum, minimum, difference between the maximum and the minimum, standard deviation, mean, median, skewness, kurtosis, zero-crossing count, energy, and peak count may be determined. In non-limiting embodiments, Fast Fourier Transform (FFT) may be applied to each window of feature data 208. Subsequently, the maximum, minimum, difference between the maximum and the minimum, standard deviation, mean, median, skewness, kurtosis, zero-crossing count, energy, and peak count may be computed on the FFT values. A mean value for all features for each half hour, resulting in 48 time slots per day, may be calculated based on the implication that different children have different average activity levels. This may result in 256 features for each of the 48 time slots.

With continued reference to FIG. 2, feature data 208 is filtered for the machine learning model inputs by focusing on time corresponding to specific activities. For example, data gathered during the sitting/quiet activity (e.g., a low movement classification) may be included, and the mean value of each feature per day may be calculated to obtain features on a daily basis which may be used as one data instance for the machine learning model. In non-limiting embodiments, 5-fold cross validation may be used to opt for the hyper-parameters in the models through grid search using F1-score as the target of the optimization. The max iteration may be tuned for a linear classifier with stochastic gradient descent (SGD), the max depth may be tuned for a Decision Tree, and the max depth and a number of estimators may be tuned for a Random Forest and Gradient Boost. Once tuned, these hyper-parameters may be fixed in a subsequent model evaluation to select one or more models for use in a production environment.

In non-limiting embodiments, feature selection 212 may be conducted to reduce the dimensions of the input features so that the machine learning models may be trained to capture the trend in the feature space efficiently. Features may be selected by comparing the importance of each feature to the average importance value of all features, and this process may be repeated until the number of selected features is fewer than a predefined number between 2 and 10. The set of features with the best training accuracy in a leave-one-participant-out cross validation may be chosen. In non-limiting embodiments, the use of context filtering may be effective for detecting hyperactive children by preventing the model from being confused by the individual difference in children's activeness.

In non-limiting embodiments, hyperactivity may be identified based on relative activity during a sitting/quiet time period, even though the activity levels may not differ much in other time periods, such as sleeping, everyday/household, exercise, school, and/or the like. With continued reference to FIG. 2, a filter may be developed that finds moments where the difference between children with and without hyperactivity would be different. This filter may replace the provided activity labels 204 in the developed machine learning model. To do so, for each test participant in a test, the activity labels may be estimated with a machine learning model that is trained using the provided activity labels 204 of the training participants.

One or more machine learning models may be used to estimate whether each user was in the sitting/quiet condition for half an hour when the data was collected. More specifically, the Random Forest model may be trained to take motion features as well as time index and day type as inputs. In non-limiting embodiments, the time index may be 48 categorical values for a day: 0 corresponds to 0:00-0:30, and 47 corresponds to 23:30-24:00. In non-limiting embodiments, the day type may be three categorical values: 0 for no school, 1 for in-person school, and 2 for virtual school. It will be appreciated that any number of categorical values may be used. Using the school day type as a feature involves the observation that school day types affect the distribution of activities. Motion features may be determined by calculating the correlation between each feature and determining whether it is sitting/quiet, then using the top five features that have a high correlation. By continuously training the machine learning model and continuously acquiring new data, and subsequently new features, the daily hyperactivity risk score 216 per subject user may be determined. The trained machine learning model may also be configured to generate a prediction 214 to classify the risk score as a hyperactive state as compared to a control state.

In non-limiting embodiments, hyperactivity may be detected automatically without provided activity labels. Such an approach may be based on developing a context filter that identifies moments where the difference between subjects with and without hyperactivity would be significant. Once the filter is developed, it replaces the provided activity labels in the already-developed ML model. The filter may be developed with data from other sensors (e.g., location, heart rate, and/or the like) and/or with existing motion-based human activity recognition models. In non-limiting embodiments, one or more machine-learning models may be trained to predict whether a subject is in sitting/quiet condition (e.g., low motion classification) for a time period (e.g., 30 minutes) when the data is collected. For example, a Random Forest model may be used that takes motion features as well as time index and day type as inputs.

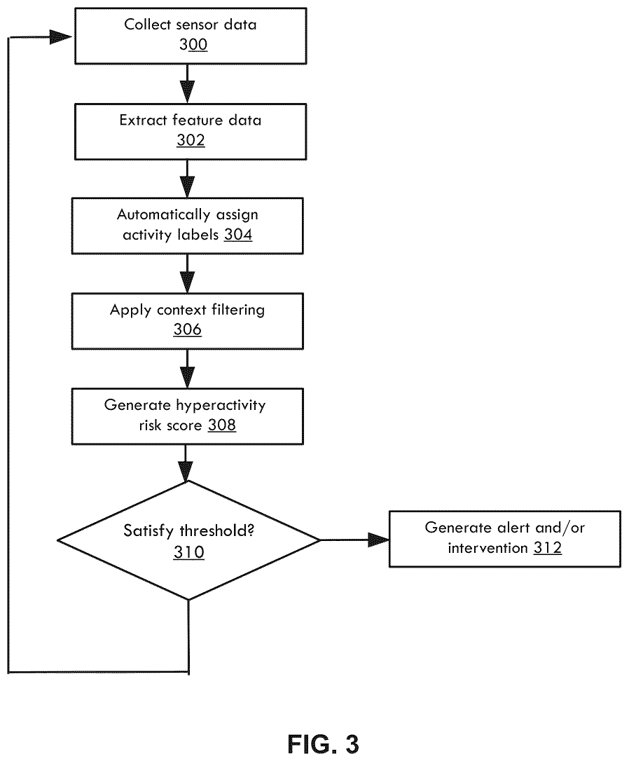

Referring now to FIG. 3, shown is a flow diagram for a method for determining hyperactivity in a subject based on sensor data according to non-limiting embodiments or aspects. The steps shown in FIG. 3 are for example purposes only. It will be appreciated that additional, fewer, different, and/or a different order of steps may be used in some non-limiting embodiments or aspects. In some non-limiting embodiments or aspects, a step may be automatically performed in response to performance and/or completion of a prior step. At step 300, sensor data may be collected from a wearable device worn by the subject user over a time period. The sensor data may include at least motion data for the subject user, such as accelerometer data, gyroscope data, geolocation data, and/or the like. At step 302, features may be extracted from the sensor data. For example, parameter values for the sensor data (e.g., accelerometer readings, spatial information, and/or the like) may be extracted and represented in a form that can be processed by a machine-learning model.

At step 304, at least one activity label of a plurality of activity labels may be automatically assigned to each feature based on at least one classification model. For example, the labels may include sitting/quiet activity, exercise, household activity, sleeping, and/or the like. At step 306, context filtering may be applied to the features based on the plurality of activity labels, resulting in filtered feature data. At step 308 a hyperactivity risk score may be generated. At step 310, the hyperactivity risk score may be compared to one or more thresholds to determine a next action. For example, if the hyperactivity risk score satisfies a threshold (e.g., meets and/or exceeds a threshold) associated with hyperactivity, the method may proceed to step 312 and an automatic alert and/or intervention step may be performed. For example, an alert may be sent to a parent/guardian and/or caregiver, a prompt and/or intervention may be sent to the wearable device, and/or the like.

Non-limiting embodiments improve upon classification systems using a Convolutional Neural Network (CNN) and Long Short-Term Memory Network (LSTM). With coarse and imprecise labels, such as provided labels from the subject and/or parent/guardian of the subject, tests of non-limiting embodiments showed that Random Forest models according to the above improve the results significantly (e.g., from 63.3% accuracy to 74.5% accuracy with the same test data in an example).

In non-limiting embodiments, the context filtering may include multiple parameters of sensor data, including geographic location (e.g., GPS), heart rate, gyroscope data, magnetometer data, and/or Bluetooth information, as examples. The data may be obtained from one or more inertial measurement units (IMU). For example, heart rate data might help improve estimations of when a subject is exercising. Geographic location data can also be informative for the model to incorporate location information (e.g., at school or at home). IMU data may be used to determine pose and/or joint orientation for determining an activity.

In non-limiting embodiments, one or more mobile applications (e.g., executed by a wearable device or an associated computing device) may proactively prompt for input while collecting data. For example, a notification may be sent to a subject and/or a parent/guardian of a subject in response to a threshold risk score or other event to prompt for an annotation of the subject's behavior (e.g., usual or unusual). The feedback received may be used as additional contextual information to assist in the predicted classification.

In non-limiting embodiments, hyperactivity scores across all the days may be combined and weighted equally. In other non-limiting embodiments, dynamic weighting algorithms based on motion-based conditional priors may be used to weight days and/or segments of time differently. In this manner, the model may learn temporal artifacts and can make a continuous prediction, rather than a snapshot analysis of each day as a segment. In non-limiting embodiments, general-purpose accelerometer datasets may be leveraged to build a baseline for a regular motion data profile that can be used as a baseline to model hyperactivity as a deviation from the baseline (e.g., treating the problem akin to an anomaly detection framework).

In non-limiting embodiments, a real-time hyperactivity risk score may be used to assist practitioners with accurate titration of medications. The process of medication titration is complex and lengthy, typically taking several weeks to months. For example, it takes more than four months for one in four pediatric patients. Providers start children at a low dose, typically not therapeutic, and gradually increase the dose until a therapeutic dose is reached. This requires several rounds of parent and teacher questionnaires and multiple office visits. Non-limiting embodiments streamline this process by providing an objective, continuous, and low-burden monitoring system to provide clinicians with rapid feedback. This is beneficial as ADHD medications, including the stimulant classes, have side effects. In non-limiting embodiments, medication titration for a subject may be automatically determined using one or more models that use, as input, one or more hyperactivity risk scores for a subject over time.

In non-limiting embodiments, an application on the wearable device may provide for the self-regulation of subjects with ADHD. For example, real-time hyperactivity scores may be automatically compared to thresholds that, in response to being satisfied, causes real-time intervention through messages, prompts, and/or or other like feedback on the wearable device.

In non-limiting embodiments, a diagnostic support tool for clinicians may be provided that uses the hyperactivity risk scores. Such a tool may include one or more GUIs to allow clinicians to inspect the risk score output by different filters, such as time and activity context, and change the filters in real-time to visualize the different results and outcomes.

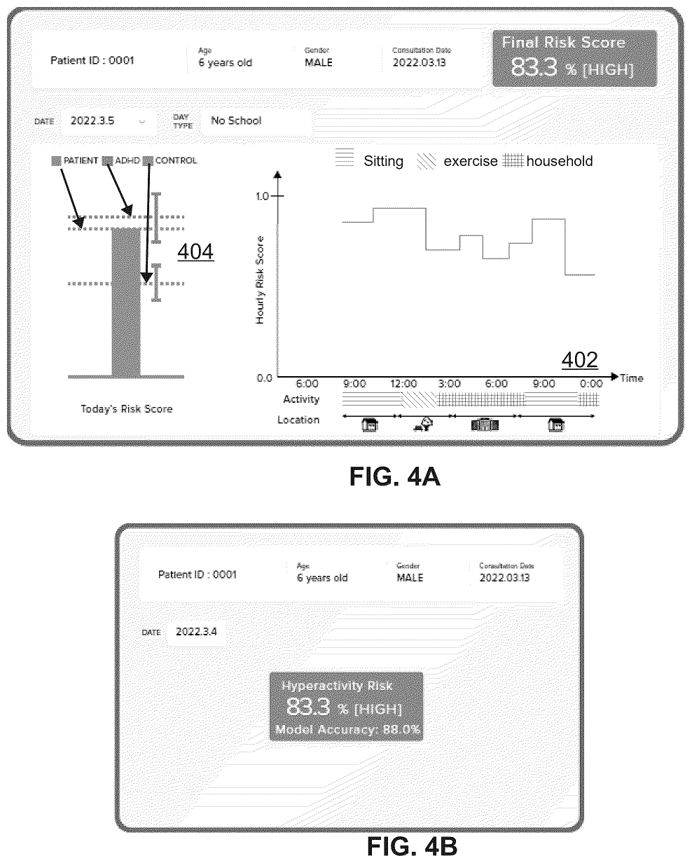

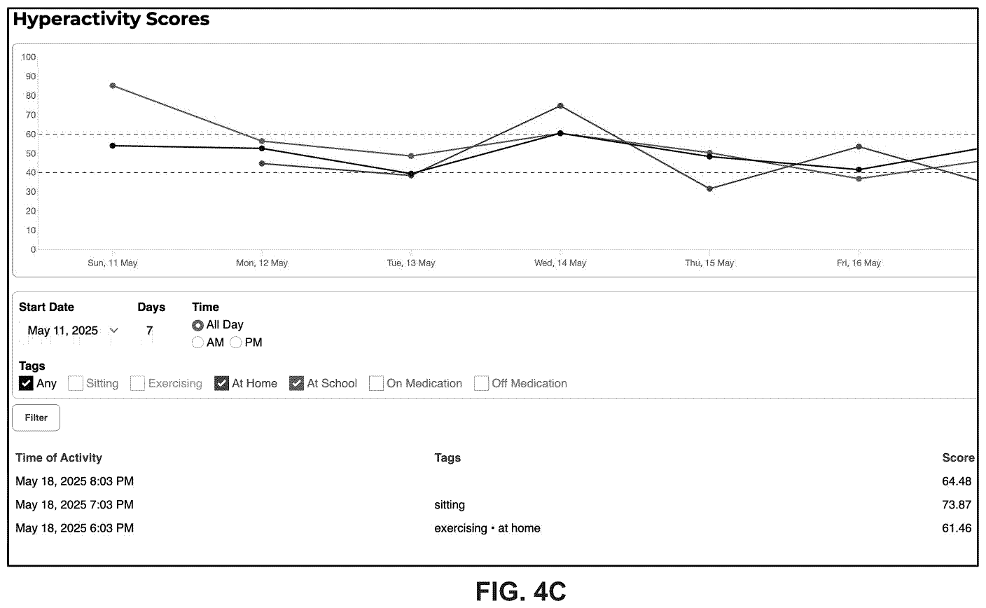

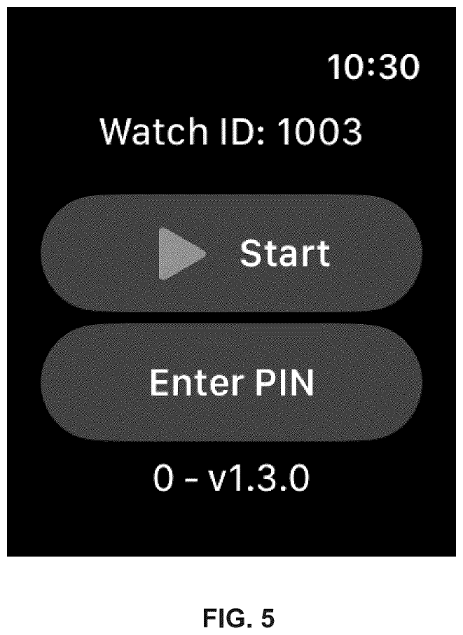

FIGS. 4A-4C and 5 show GUIs according to non-limiting embodiments or aspects. FIGS. 4A-4C show example GUIs that may be displayed on a computing device operated by a physician, clinician, parent/guardian, and/or the like. The GUIs include selectable options to facilitate a user to change subject (e.g., view data for different subjects), change context information (e.g., time and/or activity-based filters), set thresholds for automatic alerts, notifications, interventions, and/or the like, and other like tools to interact and visualize the hyperactivity data. As shown in FIG. 4A, a risk score for a time interval (e.g., one hour, 30 minutes, and/or the like) may be shown in a chart 402. The risk score may also be shown relative to thresholds 404, such as a hyperactivity threshold and a control threshold. FIG. 4C shows selectable options with filter parameters, where “at home” and “at school” labels are selected for viewing for a full day. A user may change this to include “sitting” and/or “exercising” to also display those data points. FIG. 5 shows a GUI for a wearable device. The GUI may include an identifier used to correlate to the subject and may have options for starting the application/monitoring and entering a verification input, such as a personal identification number (PIN) or the like.

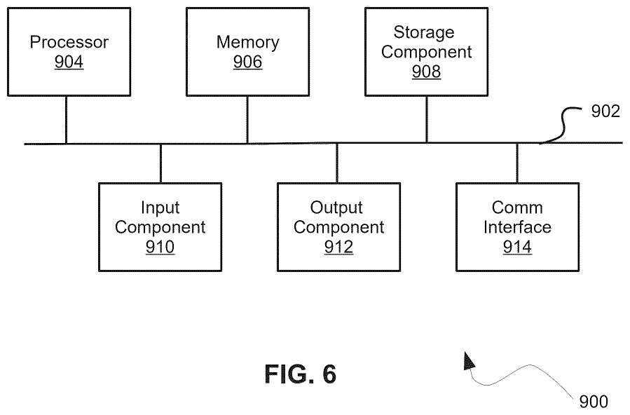

Referring now to FIG. 6, shown is a diagram of example components of a computing device 900 for implementing and performing the systems and methods described herein according to non-limiting embodiments. In some non-limiting embodiments, device 900 may include additional components, fewer components, different components, or differently arranged components than those shown in FIG. 6. Device 900 may include a bus 902, a processor 904, memory 906, a storage component 908, an input component 910, an output component 912, and a communication interface 914. Bus 902 may include a component that permits communication among the components of device 900. In some non-limiting embodiments, processor 904 may be implemented in hardware, firmware, or a combination of hardware and software. For example, processor 904 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 906 may include random access memory (RAM), read only memory (ROM), and/or another type of dynamic or static storage device (e.g., flash memory, magnetic memory, optical memory, etc.) that stores information and/or instructions for use by processor 904.

With continued reference to FIG. 6, storage component 908 may store information and/or software related to the operation and use of device 900. For example, storage component 908 may include a hard disk (e.g., a magnetic disk, an optical disk, a magneto-optic disk, a solid state disk, etc.) and/or another type of computer-readable medium. Input component 910 may include a component that permits device 900 to receive information, such as via user input (e.g., a touch screen display, a keyboard, a keypad, a mouse, a button, a switch, a microphone, etc.). Additionally, or alternatively, input component 910 may include a sensor for sensing information (e.g., a photo-sensor, a thermal sensor, an electromagnetic field sensor, a global positioning system (GPS) component, an accelerometer, a gyroscope, an actuator, etc.). Output component 912 may include a component that provides output information from device 900 (e.g., a display, a speaker, one or more light-emitting diodes (LEDs), etc.). Communication interface 914 may include a transceiver-like component (e.g., a transceiver, a separate receiver and transmitter, etc.) that enables device 900 to communicate with other devices, such as via a wired connection, a wireless connection, or a combination of wired and wireless connections. Communication interface 914 may permit device 900 to receive information from another device and/or provide information to another device. For example, communication interface 914 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 900 may perform one or more processes described herein. Device 900 may perform these processes based on processor 904 executing software instructions stored by a computer-readable medium, such as memory 906 and/or storage component 908. A computer-readable medium may include any non-transitory memory device. A memory device includes memory space located inside of a single physical storage device or memory space spread across multiple physical storage devices. Software instructions may be read into memory 906 and/or storage component 908 from another computer-readable medium or from another device via communication interface 914. When executed, software instructions stored in memory 906 and/or storage component 908 may cause processor 904 to perform one or more processes described herein. Additionally, or alternatively, hardwired circuitry may be used in place of or in combination with software instructions to perform one or more processes described herein. Thus, embodiments described herein are not limited to any specific combination of hardware circuitry and software. The term “programmed or configured,” as used herein, refers to an arrangement of software, hardware circuitry, or any combination thereof on one or more devices.

Although embodiments have been described in detail for the purpose of illustration, it is to be understood that such detail is solely for that purpose and that the disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

What is claimed is:

1. A system comprising:
at least one processor configured to:
collect sensor data from a wearable device worn by a subject user over a time period, the sensor data comprising at least motion data for the subject user;
extract features from the sensor data;
automatically assign at least one activity label of a plurality of activity labels to each feature based on at least one classification model;
apply context filtering to the features based on the plurality of activity labels resulting in filtered feature data; and
generate a hyperactivity risk score for the subject user based on at least one machine-learning model and the filtered feature data.

2. The system of claim 1, wherein the at least one processor is further configured to train the machine-learning model based on the filtered feature data.

3. The system of claim 1, wherein the at least one processor is further configured to:
determine the hyperactivity risk score for each time segment of a plurality of time segments of the time period, resulting in a plurality of risk scores; and
combine the plurality of risk scores to generate a daily hyperactivity risk score for the subject user.

4. The system of claim 1, wherein the at least one processor is further configured to display at least one graphical user interface configured to visualize the hyperactivity risk score across different contexts and time segments based on user interaction.

5. The system of claim 1, wherein the sensor data comprises the motion data and at least one of location data and heart rate data.

6. The system of claim 1, wherein the at least one processor comprises a processor of the wearable device.

7. The system of claim 1, wherein the at least one processor comprises a processor of a separate computing device.

8. A method for detecting hyperactivity in a subject user, comprising:
collecting sensor data from a wearable device worn by the subject user over a time period, the sensor data comprising at least motion data for the subject user;
extracting features from the sensor data;
automatically assigning at least one activity label of a plurality of activity labels to each feature based on at least one classification model;
applying context filtering to the features based on the plurality of activity labels resulting in filtered feature data; and
generating a hyperactivity risk score for the subject user based on at least one machine-learning model and the filtered feature data.

9. The method of claim 8, further comprising:
training the machine-learning model based on the filtered feature data.

10. The method of claim 8, further comprising:
determining the hyperactivity risk score for each time segment of a plurality of time segments of the time period, resulting in a plurality of risk scores; and
combining the plurality of risk scores to generate a daily hyperactivity risk score for the subject user.

11. The method of claim 8, further comprising:
displaying at least one graphical user interface configured to visualize the hyperactivity risk score across different contexts and temporal segments based on interaction with a user.

12. The method of claim 8, wherein the sensor data comprises the motion data and at least one of location data and heart rate data.

13. The method of claim 8, wherein collecting the sensor data comprises collecting the sensor data using a processor of the wearable device.

14. The method of claim 8, wherein collecting the sensor data comprises collecting the sensor data using a processor of a separate computing device.

15. A computer program product comprising at least one non-transitory computer-readable medium including program instructions that, when executed by at least one processor, cause the at least one processor to:
collect sensor data from a wearable device worn by a subject user, the sensor data comprising at least motion data for the subject user over a period of time;
extract features from the sensor data;
automatically assign at least one activity label of a plurality of activity labels to each feature based on at least one classification model;
apply context filtering to the features based on the plurality of activity labels resulting in filtered feature data; and
generate a hyperactivity risk score for the subject user based on at least one machine-learning model and the filtered feature data.

16. The computer program product of claim 15, wherein the at least one processor is further caused to train the machine-learning model based on the filtered feature data.

17. The computer program product of claim 15, wherein the at least one processor is further caused to:
determine the hyperactivity risk score for each time segment of a plurality of time segments of the time period, resulting in a plurality of risk scores; and
combine the plurality of risk scores to generate a daily hyperactivity risk score for the subject user.

18. The computer program product of claim 15, wherein the at least one processor is further caused to display at least one graphical user interface configured to visualize the hyperactivity risk score across different contexts and time segments based on user interaction.

19. The computer program product of claim 15, wherein the sensor data comprises the motion data and at least one of location data and heart rate data.

20. The computer program product of claim 15, wherein the at least one processor comprises a processor of the wearable device.
